(19) United States
(12) Patent Application Publication
(10) Pub. No.: US 2026/0139015 A1
(43) Pub. Date: May 21, 2026

(54) METHOD FOR PRODUCING A DEODORIZED DRY LEGUME PROTEIN

(71) Applicant: ROQUETTE FRERES, Lestrem (FR)

(72) Inventors: Aline LECOCQ, MOUVAUX (FR); Bernard CAULIER, FRETIN (FR); Christophe LAROCHE, FOUGERES (FR); Mathias IBERT, LA CHAPELLE D'ARMENTIERES (FR)

(21) Appl. No.: 19/119,310

(22) PCT Filed: Oct. 10, 2023
(86) PCT No.: PCT/EP2023/025428

§ 371 Date: Apr. 8, 2025

(30) Foreign Application Priority Data
Oct. 10, 2022 (FR) FR2210378

(51) Int. Cl. C07K 14/415; C07K 1/36

(52) U.S. Cl. CPC C07K 14/415; C07K 1/36

(57) ABSTRACT

The invention relates to a method for producing a deodorized pea protein, and to a deodorized pea protein and the use thereof for producing products requiring pressurized heat treatments, for example for producing ready-to-drink beverages or extruded products. The method comprises: providing a suspension of pea protein extract, adding peroxide to said suspension in order to form an additive-containing suspension, heat-treating the additive-containing suspension, optionally followed by rapid vacuum cooling, forming a deodorized protein solution and recovering the deodorized pea protein, wherein the weight amount of peroxide added, expressed relative to the dry weight of the pea protein extract in the suspension, ranges from 100 to 2000 pp

### FIELD OF THE INVENTION

The subject of the invention is a method for producing a protein from a pulse such as peas, particularly suitable for use in the manufacture of products requiring pressurized heat treatment, such as ready-to-drink beverages or extruded products.

### PRIOR ART

Human daily requirements for proteins are between 12 and 20% of food intake. These proteins are provided equally by products of animal origin (meat, fish, eggs, dairy products) and by plant-based food (cereals, leguminous plants, seaweed).

However, in many countries, protein intake is predominantly in the form of proteins of animal origin. And yet, numerous studies show that excessive consumption of proteins of animal origin to the detriment of plant proteins is one of the causes of increases in cancer and cardiovascular diseases.

Moreover, animal proteins have many drawbacks, both in terms of their allergenicity, notably proteins from milk or eggs, and in environmental terms, in connection with the harmful effects of intensive farming.

Thus, there is an increasing demand from manufacturers for compounds of plant origin having beneficial nutritional and functional properties without, however, having the disadvantages of compounds of animal origin.

Since the 1970s, the development of pulse plants, including in particular pea, in Europe and mainly in France, has dramatically increased as an alternative protein resource to animal proteins for animal and human food consumption. These seeds are generally non-GMOs and do not require a de-oiling step using solvents. Thus, grain legumes, also known as pulses, are distinguished from oilseed legumes such as soy.

The pea contains approximately 25% by weight of protein substances. Pea protein, predominantly pea globulin, has been extracted and utilized industrially for a great number of years. Mention may be made, as an example of a method for extracting pea protein, of patent EP1400537. In this process, the seed is milled in the absence of water (process referred to as “dry milling”) in order to obtain a flour. This flour will then be suspended in water in order to extract the protein therefrom.

Proteins from plant materials are extracted by processes that may involve various separation, purification, and treatment steps. These different steps modify their composition, color, functional and organoleptic properties, including odor.

It should be noted that odor may depend on the conditions of use of the protein ingredient: for example, if the protein is heated under pressure for the preparation of the final product, it may develop unpleasant odors, mainly sulfurous, during the manufacture of this final product if the protein has not been adequately prepared. Thus, for the production of products requiring pressurized heat treatment, such as ready-to-drink beverages, or extruded products, it may be necessary to provide proteins capable of not developing these odors.

Methods for deodorizing plant proteins have already been described, including methods for deodorizing pea proteins. The overview Pua et al, Ingredients, Processing, and Fermentation: Addressing the Organoleptic Boundaries of Plant-Based Dairy Analogues. Foods, 2022, 11, 875 cites various documents describing the deodorization of plant proteins. It is reported that, for peas, the odor of the protein could be improved using different methods: by applying a prior dehulling of the pea, by alkaline treatment during soaking before protein extraction, by washing the flour with organic solvents or by treatment with supercritical CO₂ in combination with ethanol. Also, WO2011/124862 A1 on behalf of the Applicant describes the production of functionalized proteins; according to a preferred embodiment, the method comprises a step of cooling heated proteins which is carried out by applying a significant vacuum, so as to maximize deodorization of the protein.

The applicant has succeeded in developing a method for producing proteins from pulses such as peas, suitable for use in the production of products requiring pressurized heat treatment, without developing unpleasant odors and in particular without sulfurous odors. Surprisingly, the proteins produced can also retain identical good functional properties (solubility) in a preferred embodiment.

The invention is described below.

### SUMMARY OF THE INVENTION

The invention relates to a method for producing a deodorized pulse protein, preferably from peas, which comprises:
providing a suspension of pulse protein extract, preferably from peas,
adding peroxide to said suspension in order to form an additive-containing suspension,
heat-treating the additive-containing suspension, optionally followed by rapid vacuum cooling, in order to form a deodorized protein solution,
recovering the deodorized pulse protein, preferably pea protein,
   wherein the weight amount of peroxide added, expressed relative to the dry weight of the pulse protein extract, preferably pea protein, in the suspension, ranges from 100 to 2000 ppm.

Another object further relates to deodorized pulse protein, preferably pea protein, obtainable by the process according to the invention.

### DESCRIPTION OF THE FIGURE

FIG. 1 shows the molecular profiles of different pea protein samples. It consists of the image of electrophoresis gels obtained by SDS-PAGE under non-reducing conditions, after migration and staining, with the molecular mass expressed in kDa shown on the left-hand side of the FIGURE.

### DETAILED DESCRIPTION OF THE INVENTION

The invention relates to a method for producing a deodorized pulse protein, preferably pea protein.

Dry legumes are also known to those skilled in the art as “pulses”. Pulses differ from leguminous oilseeds such as soy in their low total lipid content. This total lipid content relative to the dry matter of the seed is generally less than 10%, often less than 5%. This total lipid content can be determined by the AOAC 996.06 method. Pulses can be those listed in Codex Standard 171-1989, as revised in 1995 and amended in 2012. The pulses listed are as follows:
Phaseolus spp. beans (except Phaseolus mungo L. syn. Vigna mungo (L.) Hepper and Phaseolus aureus Roxb. syn. Phaseolus radiatur L., Vigna radiata (L.) Wilczek);
Lentils of culinaris Medic. Syn. Lens esculenta Moench.;
Peas of Pisum sativum L.;
Chickpeas of Cicer arientinum L.;
Beans of Vicia faba L. (also known as faba beans);
Cowpeas (black-eyed peas) of Vigna unguiculata (L.) Walp., Syn. Vigna sesquipedalis Fruwh., Vigna sinensis (L.) Savi exd Hassk.

However, other seeds meeting the definition of pulses according to the invention can also be cited, such as lupins or mung beans.

The invention further relates to a method for producing a deodorized pulse or faba bean protein, preferably pea protein.

The term “pea” is considered herein in its broadest sense and including in particular:
all varieties of “smooth pea” and “wrinkled pea”, and
all the mutant varieties of “smooth pea” and of “wrinkled pea”, regardless of the uses for which said varieties are usually intended (human food, animal feed and/or other uses).

The term “peas” in the present application includes pea varieties belonging to the Pisum genus and more particularly to the species sativum and aestivum. Said mutant varieties are in particular those called “mutants r”, “mutants rb”, “mutants rug 3”, “mutants rug 4”, “mutants rug 5” and “mutants lam” as described in the article by C-L HEYDLEY et al., entitled “Developing novel pea starches”, Proceedings of the Symposium of the Industrial Biochemistry and Biotechnology Group of the Biochemical Society, 1996, pp. 77-87. Since the 1970s, peas have been the most widely grown protein-rich pulse plant in Europe, especially in France, not only as a source of protein for animal feed, but also for human consumption. Pea proteins consist of three main classes of proteins: globulins, albumins and so-called insoluble proteins.

For the sake of simplicity, the following description describes the pea method in detail, but it is made clear that the invention is applicable to all the above-mentioned pulses, simply by replacing the term “pea” with “pulse” or with at least one of the above-mentioned pulse sources, such as faba beans.

The term “pea protein extract” should be understood in the present application to mean a composition extracted from peas comprising primarily polypeptide chains, or proteins, consisting of a sequence of amino acid residues bonded to one another by peptide bonds. The pea protein extract can be extracted by any type of process, dry or wet. The pea protein extract can be selected from pea protein isolate or pea protein concentrate. The pea protein extract may comprise different classes of proteins. Preferably, the pea protein of the invention comprises mainly globulins. The term “deodorized pea protein” refers to a pea protein with a reduced, non-unpleasant odor. In particular, this means that, compared with non-deodorized pea protein produced by a method that differs only in the absence of added peroxide, the deodorized pea protein of the invention has a lesser odor when subjected to a pressurized heat treatment step. Preferably, after heat treatment according to TEST A as described in more detail in the Examples section, the deodorized pea protein has a reduced sulfurous odor or no sulfurous odor at all. For example, this TEST A can be carried out 29 days after pea protein production. The Examples section lists tests showing the deodorization of pea proteins according to the invention.

The method of the invention comprises providing a suspension of pea protein extract. Generally speaking, the suspension can have a dry matter content ranging from 1 to 50%, for example from 5 to 35%, particularly from 10 to 25%. The suspension of pea protein extract is generally an aqueous suspension. The dry matter of the suspension of pea protein extract generally consists of pea protein extract as defined below. The pH of the suspension of pea protein extract can vary widely. It can range from 1 to 9, generally from 2 to 6, for example from 4.5 to 5.5. To perform the pH adjustment, any type of acid and/or base, organic or inorganic, or mixtures thereof, can be added to the suspension. Examples of acids that can be used include hydrochloric acid, sulfuric acid, citric acid or mixtures thereof. As an example of a base, mention may be made of sodium hydroxide, potash or lime and the mixtures thereof. The base or acid is generally added via an aqueous solution.

The pea protein extract can be of any type and can be extracted by any dry or wet process. In one embodiment, the pea protein extract of the suspension is obtained by isoelectric precipitation. A pea protein extract obtained by isoelectric precipitation is conventionally obtained by a method which comprises preparing an aqueous suspension of pea flour, solid-liquid separation of the suspension to obtain a soluble fraction and an insoluble fraction, a step of isoelectric precipitation of the proteins comprised in the soluble fraction so as to form a pea protein extract solution, and separation of the precipitated proteins in order to recover a suspension of pea protein extract. Pea flour suspension can be produced by dry grinding peas, previously dehulled, to produce a flour which is then suspended in water. Alternatively, the pea flour suspension can be produced by wet-grinding dehulled peas. As an example of a method for producing pea protein extract using wet grinding, reference may be made to document WO2019/053387 A1 in the name of the Applicant.

According to the invention, the protein richness of pea protein extract and deodorized pea protein is N6.25, calculated by the Dumas method.

The N6.25 protein content of the pea protein extract in the suspension supplied, expressed on a dry weight basis, is for example 60% or more, advantageously 80% or more, for example ranges from 80 to 95%, in particular ranges from 80 to 90%. Preferably, the pea protein extract is a pea protein isolate. According to the invention, protein isolate is understood to mean a pea protein having a protein content of 80% or more.

Although a pea protein (such as the pea protein extract useful in the invention and the deodorized pea protein of the invention) is primarily defined by its protein content, it obviously generally comprises other non-protein minority constituents, such as starch, lipids, fibers, sugars and/or minerals. Generally, the total starch content in the pea protein extract ranges from 0% to 20%, for example from 0% to 10%, especially from 0.5% to 5%. This total starch content can be measured using the AOAC 996.11 method. Generally, the total fiber content may range from 0% to 20%, for example from 1% to 18%, in particular from 2% to 10%. This content can be determined by AOAC Method 2017.16. Generally, the total lipid content is from 0% to 15%, for example from 1% to 10%. The total lipid content can be determined by AOAC Method 996.06 using acid hydrolysis. The sugar content may range from 0% to 10%, generally from 0.5% to 5%. The sugar content can be determined by high performance liquid chromatography (HPLC). The mineral content can be determined by determining the ash rate. All the above contents are expressed relative to the dry weight of the pea protein extract.

One advantage of the invention is that pea protein deodorized by the method of the invention can have an unaltered molecular profile compared to non-deodorized pea protein, made by a method that differs only in the absence of added peroxide. Deodorized pea protein can also have the same composition as the pea protein extract supplied and described above. The N6.25 protein content of the deodorized pea protein according to the invention, as well as its minority constituents, can thus be in the same proportions as those described above.

The method comprises adding peroxide to the suspension of pea protein extract. The peroxide is preferably hydrogen peroxide.

Hydrogen peroxide can be introduced in the form of an aqueous solution of hydrogen peroxide comprising, based on its total weight, from 1 to 95% by weight hydrogen peroxide, for example from 5 to 50%. According to the invention, the weight amount of peroxide added, expressed relative to the dry weight of the pea protein extract in the suspension, ranges from 100 to 2000 ppm.

The amount of peroxide added by weight, expressed relative to the dry weight of pea protein extract in the suspension, can range from 110 to 1000 ppm, for example from 120 to 800 ppm, for example from 130 to 600 ppm. Advantageously, the amount of peroxide added by weight, expressed relative to the dry weight of pea protein extract in the suspension, can range from 150 to 500 ppm, for example from 200 to 400 ppm, or even from 200 to 350 ppm.

According to this preferred embodiment, and as shown in the Examples section below, it has been observed that even with these very low quantities of added peroxide, the method surprisingly enables the production of deodorized pea protein without even modifying the molecular profile, nor altering functionalities (solubility).

This addition step can be very rapid, lasting a few seconds, or last a few minutes, and an additive-containing suspension is formed at the end of this step. This additive-containing suspension can have a dry matter content ranging from 1 to 50%, for example from 5 to 35%, particularly from 10 to 25%.

After the addition step and before the heat treatment step, a mixing step can optionally be carried out. It is also possible to carry out an optional storage step. These optional steps can take from a few minutes to a few hours.

The method of the invention comprises a step of heat treatment of the additive-containing suspension. Advantageously, the heat treatment is carried out at a temperature ranging from 80 to 160° C., preferably from 100 to 150° C.

According to one embodiment, the pH of the additive-containing pea protein suspension, before heat treatment, ranges from 6 to 7.5. The pH can be adjusted using the organic or inorganic acids and bases mentioned above.

The heat treatment step of the additive-containing suspension of the method may be followed by rapid vacuum cooling. Preferably, the vacuum level of the rapid cooling step is set so that the temperature of the heat-treated solution is cooled by at least 10° C., for example to a temperature of between 6° and 80° C. Depending on the method, a plurality of heat treatments and/or rapid coolings can be carried out.

At the end of this heat treatment step, possibly followed by rapid cooling, a deodorized pea protein solution is obtained. Deodorized pea protein is recovered. Preferably, the method comprises a drying step for the deodorized protein solution. Thus, the deodorized protein solution is dried, preferably by atomization, to form the deodorized pea protein in solid form. The deodorized protein is advantageously in powder form.

According to one embodiment, the deodorized pea protein has a solubility in water at pH 7, determined according to test B described in the Examples section, greater than or equal to 30%, for example ranging from 40 to 80%.

Advantageously, the deodorized pea protein comprises an amount of dihydrogen sulfide of less than 50 ppb, or even less than 30 ppb. This quantity can be measured by solid-phase microextraction followed by gas chromatography-mass spectrometry analysis. The operating details of such a method can be found in TEST C, described in the Examples section.

The method can be a batch process or continuous process.

The invention further relates to a deodorized pea protein obtainable by the method of the invention.

The invention further relates to the use of deodorized pea protein obtained according to the method of the invention for the production of products requiring pressurized heat treatments, for example for the production of ready-to-drink beverages or extruded products.

Generally, the resulting pea protein can be used in food products and beverages that may include it in an amount up to 100% by weight relative to the total dry weight of the food or beverage product, for example in an amount ranging from about 1% by weight to about 80% by weight relative to the total dry weight of the food or beverage product. All intermediate amounts (that is, 2%, 3%, 4% . . . 77%, 78%, 79% by weight relative to the total weight of the food or beverage product) can be used, as well as all the intermediate ranges based on these quantities. The food or beverage products that can be concerned comprise baked products; sweet baked products (including, but not limited to, rolls, cakes, pies, pastries, and cookies); pre-made sweet baking mixtures for preparing sweet baked products; pie fillings and other sweet fillings (including, but not limited to, fillings for fruit pies and fillings for nut pies, such as fillings for pecan pies, as well as fillings for cookies, cakes, pastries, confectionery products and similar products, such as fillings for fat-based cream); desserts, gelatins and puddings; frozen desserts (including, but not limited to, frozen dairy desserts such as ice cream-including ordinary ice cream, soft serve ice cream and all other types of ice cream- and non-dairy frozen desserts such as non-dairy ice cream, sorbet and similar products); carbonated beverages (including, but not limited to, carbonated soft drinks); non-carbonated beverages (including, but not limited to, non-carbonated soft drinks such as flavored waters, fruit juices and sweetened tea or coffee-based beverages); beverage concentrates (including, but not limited to, liquid concentrates and syrups as well as non-liquid “concentrates,” such as freeze-dried and/or powdered preparations); yogurts (including, but not limited to, high-fat, reduced-fat and fat-free dairy yogurts, as well as non-dairy and lactose-free yogurts); snack bars (including, but not limited to, cereal bars, nut bars, and/or fruit bars); bread products (including, but not limited to, yeasted and unyeasted breads, yeasted breads and undyed breads such as soda breads, breads comprising any type of wheat flour, breads composed of any type of non-wheat flour (such as potato, rice and rye flour), gluten-free bread); bread mixtures for preparing bread products; sauces, syrups and vinaigrettes; sweetened spreads (including, but not limited to, jellies, jams, butters, nut spreads and other preserves, preserves and other spreadable preserves); confectionery products (including, but not limited to, jelly beans, soft candies, hard candies, chocolates and gums); sugar-coated and not sugar-coated breakfast cereals (including, but not limited to, extruded breakfast cereals, flaked breakfast cereals and puffed breakfast cereals); and coating compositions for cereals intended for the preparation of sweet breakfast cereals. Other types of food and beverages that are not mentioned herein but which conventionally comprise one or more proteins can also be envisaged in the context of the present invention. In particular, animal feed (such as pet food) is explicitly envisaged. It can also be used, optionally after texturing by extrusion, in products similar to meat such as emulsified sausages or plant-based hamburgers. It can also be used in egg replacement formulations.

The food or beverage product can be used in specialized nutrition, for specific populations, for example for babies or infants, elderly people, athletes, or in clinical nutrition (for example, feeding by probe or enteral nutrition).

The deodorized pea protein can be used as a single source of proteins, but can also be used in combination with other plant or animal proteins.

The term “plant protein” denotes all the proteins derived from cereals, oleaginous plants, leguminous plants and tuberous plants, as well as all the proteins derived from algae and microalgae or fungi, used alone or as a mixture, selected from the same family or from different families.

In the present application, the term “cereals” refers to plants cultivated from the family of grasses producing edible grains, for example wheat, rye, barley, corn, sorghum or rice. Grain are often ground in flour form, but are also provided in the form of cereals and sometimes in the form of whole plants (forage crops). Tubers may be carrot, cassava, konjac, potato, Jerusalem artichoke, sweet potato.

The animal protein can, for example, be egg or milk proteins, such as whey proteins, casein or caseinate proteins. The pea protein composition can thus be used in association with one or more of these proteins or amino acids in order to improve the nutritional properties of the final product, for example to improve the PDCAAS or to provide or modify other functionalities.

The invention will now be described in particular embodiments in the Examples section, which in no way limit the scope of the present invention.

### EXAMPLES

### Methods

### Test A: Determining the Pea Protein Odor

The protein powder obtained is mixed with demineralized water at room temperature at a concentration of 5% by dry weight. 5L of solution is prepared in a beaker fitted with an Ultraturax immersion blender for a few minutes to obtain a homogeneous mixture.

The homogeneous mixture, preheated to 80° C. by passing through a tubular heat exchanger, is sent by a centrifugal pump to a heat treatment unit equipped with an Armfield indirect tubular heat exchanger through which steam circulates. The heat treatment scale applied to the suspension is 130° C. for 30 seconds. The suspension is then immediately cooled to around 30° C. by a tubular cooler.

The sulfurous odor of the suspension is evaluated before preheating and after cooling by a panel of 5 people experienced and trained in evaluating the odor of pea proteins.

### Test B: Measuring Solubility in Water at pH 7

This measurement is based on the dilution of the sample in distilled water, its centrifuging and the analysis of the supernatant.

### Procedure:

Introduce 150 g of distilled water into a 400 ml beaker at 20° C.±2° C., mix with a magnetic stirrer bar, and add precisely 5 g of the sample to be tested.

Adjust the pH to the desired value with 0.1 N NaOH or HCl (pH 7), or do not adjust it.

Complete water content at 200 g.

Mix for 30 minutes at 1000 rpm and centrifuge for 15 minutes at 3000 g.

Collect 25 g of the supernatant.

Introduce into a previously dried and tared crystallizer.

Place in an oven at 103° C.±2° C. for 1 hour.

Then place in a desiccator (with desiccant) to cool to ambient temperature and weigh.

The soluble dry matter content, expressed in % by weight, is given by the following formula:

[(m1−m2)×(200+P)×100]/(P1×P)=% of solubility
where:
P=weight, in g, of the sample=5 g
m1=weight, in g, of the crystallizer after drying
m2=weight, in g, of the empty crystallizing dish
P1=weight, in g, of the sample collected=25 g

Test C: Determining the Quantity of Dihydrogen Sulfide

An exact mass of a pea protein sample is dissolved. The preparations are subjected to solid-phase microextraction (SPME), and desorption from the support is carried out in the injector of a Shimadzu 2010 chromatograph equipped with a PDMS chromatography column. The analyses are carried out by coupled gas chromatography-mass spectrometry GC-MS (Shimadzu QP2010+ mass spectrometer) and the ionization method is electron impact (70 eV). The dihydrogen sulfide has a retention time measured at 1.26 min (characteristic ions: 33 and 34).

Coloration parameters L, a, and b may be determined using a spectrophotometer, via the CIE Lab model.

### Control Tests

The control protocol below was carried out:

Smooth yellow pea flour was mixed with water to form a suspension with 20% dry matter

The soluble and insoluble parts (fibers, starch) were separated by centrifugation

The soluble part containing proteins (approx. 7% dry matter) was transferred into a stirred tank equipped with a double jacket

Acidification to pH 5 was carried out with HCl and protein flocculation (by heating to approx. 70° C.)

Flocculated proteins (mainly globulins) were extracted in a centrifugal decanter

Those flocculated proteins corresponding to a suspension of pea protein extract were recovered and diluted with water at room temperature in a stirred tank

The suspension with 13% dry matter was carried out with a propeller shaker for 30 minutes

1N sodium hydroxide was added to neutralize, still with stirring, until a pH of 7 was obtained

Heat treatment was applied by direct steam injection for 10 s at 130° C., then flash at 70° C.

Spray drying was performed in a NUBILOSA pilot atomizer: drying temperature 190-195° C.; product outlet temperature 90-95° C.

### Tests According to the Invention

The below protocol according to the invention was carried out:

Smooth yellow pea flour was mixed with water to form a suspension with 20% dry matter

The soluble and insoluble parts (fibers, starch) were separated by centrifugation

The soluble part containing proteins (approx. 7% dry matter) was transferred into a stirred tank equipped with a double jacket

Acidification to pH 5 was carried out with HCl and protein flocculation (by heating to approx. 70° C.)

Flocculated proteins (mainly globulins) were extracted in a centrifugal decanter

A suspension of those flocculated proteins corresponding to a suspension of pea protein extract was recovered and diluted with water at room temperature in a stirred tank to which a solution of hydrogen peroxide (5% by weight of hydrogen peroxide) was added

The additive-containing suspension with 13% dry matter was formed with a propeller shaker for 30 minutes

1N sodium hydroxide was added to neutralize, still with stirring, until a pH of 7 was obtained

Heat treatment was applied by direct steam injection for 10 s at 130° C., then flash at 70° C.

Spray drying was performed in a NUBILOSA pilot atomizer: drying temperature 190-195° C.; product outlet temperature 90-95° C.

Based on this protocol, several tests were carried out using different quantities of hydrogen peroxide solution.

Each protocol was used with two different batches of peas (batches A and B). For the control protocol, the batch A test is listed as test 1 and the batch B test as test 3. For the protocol according to the invention, the various tests with batch A are listed under the references test 2-XXX, and the various tests with batch B are listed under the references test 4-XXX with XXX representing the weight quantity of hydrogen peroxide involved in the test, expressed in ppm of hydrogen peroxide relative to the dry weight of the pea protein extract.

### Table 1 Below Lists:

the batch of peas used
the test reference
the weight quantity of hydrogen peroxide used, expressed as pure weight in relation to the quantity of pea protein extract dry matter in the suspension
protein richness expressed by dry weight of the sample
dry weight
sulfurous odor determined by TEST A before and after heat treatment (HT).
The test A evaluations were conducted 5 days (5 d) after production and 29 days (29 d) after production
solubility according to test B
The amount of dihydrogen sulfide according to TEST C.

### Table 2 Below Lists:

the batch of peas used
the test reference
the weight quantity of hydrogen peroxide used, expressed as pure weight in relation to the quantity of pea protein extract dry matter in the suspension
protein richness expressed by dry weight of the sample
dry weight.
The L, a and b color parameters of the powder
the sulfur odor according to TEST A after heat treatment (HT); the evaluation of test A was carried out 3 days (3 d) after production.

### Example 5 According to the Invention

Further tests were carried out according to the following protocol:

Smooth yellow pea flour was mixed with water to form a suspension with 20% dry matter

The soluble and insoluble parts (fibers, starch) were separated by centrifugation

The soluble part containing proteins (approx. 7% dry matter) was transferred into a stirred tank equipped with a double jacket

Acidification to pH 5 was carried out with HCl and protein flocculation (by heating to approx. 70° C.)

Flocculated proteins (mainly globulins) were extracted in a centrifugal decanter

A suspension of those flocculated proteins corresponding to a suspension of pea protein extract was recovered and diluted with water at room temperature in a stirred tank to which a solution of hydrogen peroxide (5% by weight of hydrogen peroxide) was added

The additive-containing suspension with 13% dry matter was formed with a propeller shaker for 30 minutes

1N sodium hydroxide was added to neutralize, still with stirring, until a pH of 6.3-6.5 was obtained

Heat treatment was applied by direct steam injection for 10 s at 120° C., then flash at 70° C.

Spray drying was performed in a NUBILOSA pilot atomizer: drying temperature 190-195° C.; product outlet temperature 90-95° C.

The quantities of hydrogen peroxide are identical to those used in test 4.

### Results Analysis

Table 1 below shows the results obtained for proteins from Trials 1 and 2.

TABLE 1

| | | | | | Odor | Odor | Odor | Odor | | |
|---|---|---|---|---|---|---|---|---|---|---|
| Pea | | H2O2 | N6.25 | SC | before HT | after HT | before HT | after HT | Sol | H2S |
| batch | Test | (ppm) | (%) | (%) | (5 d) | (5 d) | (29 d) | (29 d) | (%) | (ppb) |
| A | 1 | 0 | 85.4 | 95.2 | none | strong | none | strong | 75 | 79 |
| | 2-220 | 220 | 85.8 | 95.5 | none | slight | none | none | 73 | 19 |
| | 2-275 | 275 | 85.7 | 95.5 | none | slight | none | none | 70 | 24 |
| | 2-315 | 315 | 85.4 | 95 | none | none | none | none | 71 | 24 |
| | 2-920 | 920 | 84.3 | 95.7 | none | none | none | none | 63 | nd |

Table 1 shows that the use of hydrogen peroxide in the method does not affect protein richness. Tests 2-220, 2-275 and 2-315 also show that the functionalities (solubility) of the pea proteins are unchanged compared to the pea protein in control test 1.

When not heat-treated, the control pea protein did not show any unpleasant sulfurous odor, but a strong odor appeared when subjected to pressurized heat treatment, 5 days after protein production and even 29 days later. On the contrary, even with the lowest amounts of peroxide, the method of the invention using hydrogen peroxide delivers proteins whose sulfurous odor is greatly reduced, or even eliminated, when the pea protein of the invention is heat-treated five days after production. Twenty-nine days after production, none of the proteins according to the invention showed any unpleasant odor when subjected to pressurized heat treatment.

Table 2 below shows the results obtained for proteins from Trials 3 and 4 using a different batch of peas (batch B) than in Examples 1 and 2 (batch A).

TABLE 2

| Pea | | H2O2 | N6.25 | | | | | Odor after |
|---|---|---|---|---|---|---|---|---|
| batch | Test | (ppm) | (%) | SC (%) | L | a | b | HT (3 d) |
| B | 3 | 0 | 85.7 | 96.8 | 85.7 | 1.3 | 16 | strong |
| | 4-180 | 180 | 84.8 | 95.8 | 85.6 | 1.3 | 15.0 | slight |
| | 4-240 | 240 | 84.8 | 95.7 | 87 | 1.4 | 12.5 | none |
| | 4-280 | 280 | 84.8 | 96.2 | 85.8 | 1.7 | 14.4 | none |
| | 4-315 | 315 | 85.2 | 95.7 | 85.5 | 1.9 | 14.4 | none |
| | 4-920 | 920 | 84.7 | 95.6 | 84.2 | 2.9 | 14.7 | none |

Table 2 shows that, to obtain a deodorized pea protein, the quantities of hydrogen peroxide are very similar whatever batch of peas is used, albeit slightly different. After only three days of production, no odor was noted after heat treatment of the protein made from pea batch B using 240 ppm hydrogen peroxide (test 4-240); for the test using batch A and using 275 ppm hydrogen peroxide (test 2-275), the deodorized pea protein exhibited a slight sulfurous odor after heat treatment carried out 5 days after the protein was produced. The conclusions of tests 3 and 4 therefore remain very close to those obtained for tests 1 and 2 as far as deodorization is concerned.

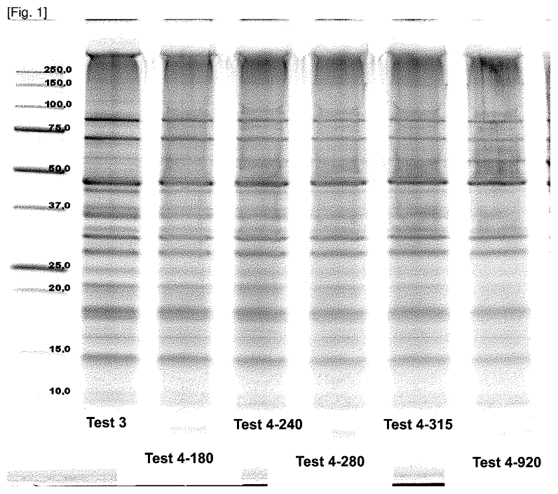

SDS-PAGE electrophoresis analysis under non-reducing conditions shown in FIG. 1 also demonstrates that the deodorized pea protein sample manufactured with a method using a hydrogen peroxide dose of 920 ppm exhibits a slightly altered protein structure compared with the control. For this sample, a band appears at 60 kDa, corresponding to the legumin band; the bands of the α and β-legumin subunits are not very present (40 and 20 kDa respectively). Contrarily, when peroxide levels reach 315 ppm, the protein structure remains unchanged, as demonstrated by the SDS PAGE electrophoresis analyses, which are identical to those of the control: the legumin band is not present (60 kDa), and 2 bands appear at around 40 and 20 kDa respectively, demonstrating that the α and β-legumin subunits are dissociated, as in the case of the control.

As for the proteins obtained in test 5, they have a more neutral odor and

no sulfurous notes, like those in test 4.

1. A method for producing a deodorized pulse protein, wherein it comprises:
providing a suspension of pulse protein extract,
adding peroxide to said suspension in order to form an additive-containing suspension,
heat-treating the additive-containing suspension, optionally followed by rapid vacuum cooling, in order to form a deodorized protein solution,
recovering deodorized pea protein,
wherein the weight amount of peroxide added, expressed relative to the dry weight of the pulse extract in the suspension, ranges from 100 to 2000 ppm.

2. The production method according to claim 1, wherein the pulse is chosen from pea and faba bean.

3. The production method according to claim 1, wherein the pulse is pea.

4. The production method according to claim 1, wherein the mass quantity of added peroxide, expressed relative to the dry mass of pulse protein extract in the suspension, ranges from 150 to 500 ppm, for example from 200 to 400 ppm, or even 200 to 350 ppm.

5. The production method according to claim 1, wherein the heat treatment is carried out at a temperature ranging from 80 to 160° C., preferentially from 100° C. to 150° C.

6. The production method according to claim 5, wherein the vacuum in the rapid vacuum cooling step is set so that the temperature of the heat-treated solution is cooled by at least 10° C., for example to a temperature from 60 to 80° C.

7. The production method according to claim 1, wherein the production method comprises a step of drying the deodorized protein solution, preferably by atomization.

8. The production method according to claim 1, wherein the deodorized protein is in powder form.

9. The method according to claim 1, wherein the peroxide is a hydrogen peroxide.

10. The method according to claim 1, wherein the pulse protein extract of the suspension is obtained by isoelectric precipitation.

11. The method according to claim 1, wherein the pH of the suspension of pulse protein extract ranges from 2 to 6, for example from 4.5 to 5.5.

12. The method according to claim 1, wherein the pH of the additive-containing pulse protein suspension, prior to heat treatment, ranges from 6 to 7.5.

13. The method according to claim 1, wherein the protein richness N6.25 according to the Dumas method of the pulse protein extract in the suspension provided, expressed on a dry weight basis, is 60% or more, advantageously is 80% or more, for example ranges from 80 to 95%, in particular ranges from 80 to 90%.

14. The method according to claim 1, wherein the deodorized pulse protein comprises an amount of dihydrogen sulfide of less than 50 ppb, or even less than 30 ppb.

15. A deodorized pulse protein that can be obtained by the method according to claim 1.

16. A use of a pulse protein according to claim 15 for the manufacture of products requiring pressurized heat treatment, for example for the manufacture of ready-to-drink beverages or extruded products.
